(19) United States
(12) Patent Application Publication
(10) Pub. No.: US 2010/0304391 A1
(43) Pub. Date: Dec. 2, 2010

(54) METHODS FOR ASSESSMENT AND TREATMENT OF DEPRESSION VIA UTILIZATION OF SINGLE NUCLEOTIDE POLYMORPHISMS ANALYSIS

(76) Inventor: Jay L. Lombard, New City, NY (US)

Correspondence Address: SHAY GLENN LLP, 2755 CAMPUS DRIVE, SUITE 210, SAN MATEO, CA, 94403, US

(21) Appl. No.: 12/790,262

(22) Filed: May 28, 2010

Related U.S. Application Data
(60) Provisional application No. 61/217,338, filed on May 29, 2009.
(60) Provisional application No. 61/325,098, filed on Apr. 16, 2010.

(51) Int. Cl. C12Q 1/68

(52) U.S. Cl. 435/6

(57) ABSTRACT

Described herein are assays, kits and methods for treating depression, including the diagnosis and treatment of depression based on the determination of genetic predispositions towards inhibition or enhancement of Ca2+/calmodulin-dependent protein kinase II (CaMKII). For example, described herein are methods and kits (including assays) for determining if one or more gene in an excitatory or inhibitory pathway for modulating CaMKII activity or expression is likely to be inhibited or enhanced by an SNP. Also described are methods and kits (including assays) for prescribing treatment based on the identification of SNPs that may modulate CaMKII.

### CROSS REFERENCE TO RELATED APPLICATIONS

This patent claims priority to U.S. Provisional Patent Application No. 61/217,338, titled “SYSTEM AND METHOD FOR DIAGNOSIS AND TREATMENT OF COMMON MENTAL HEALTH COMPLAINTS,” filed on May 29, 2009, and U.S. Provisional Patent Application No. 61/325,098, titled “MODULATION OF SEROTONIN REUPTAKE BASED ON GENOTYPE TO TREAT DEPRESSION,” filed on Apr. 16, 2010. These patent applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices, methods, and systems described herein relate to the diagnosis and treatment of depression, and particularly to the treatment of depression based on the determination of genetic predispositions towards inhibition or enhancement of Ca2+/calmodulin-dependent protein kinase II (CaMKII).

### BACKGROUND OF THE INVENTION

Between 5-10% of adults worldwide suffer from depression.

The economic costs to society and the personal costs to individuals and families, associated with depression are enormous. Within a 15-month period after having been diagnosed with depression, sufferers are four times more likely to die as those who do not have depression. Almost 60% of suicides have their roots in major depression, and 15% of those admitted to a psychiatric hospital for depression eventually kill themselves. In the U.S. alone, the estimated economic costs for depression in 1990 exceeded $44 billion. The World Health Organization estimates that major depression is the fourth most important cause worldwide of loss in disability-adjusted life years, and will be the second most important cause by 2020.

A variety of pharmacologic agents are available for the treatment of depression. Significant success has been achieved through the use of serotonin reuptake inhibitors (SRIs), norepinephrine reuptake inhibitors (NERIs), combined serotonin-norepinephrine reuptake inhibitors (SNRIs), monoamine oxidase inhibitors (MAOIs), phosphodiesterase-4 (PDE4) inhibitors or other compounds. However, even with these options available, many patients fail to respond, or respond only partially to treatment. Additionally, many of these agents show delayed onset of activity, so that patients are required to undergo treatment for weeks or months before receiving benefits. Most currently available antidepressants take 2-3 weeks or more to elicit a response.

Traditional therapies can also have significant side effects. For example, more than a third of patients taking SRIs experience sexual dysfunction. Other problematic side effects include gastrointestinal disturbances, often manifested as nausea and occasional vomiting, agitation, insomnia, weight gain, onset of diabetes,

Thus, there remains a need for the development of improved therapies for the treatment of depression and/or other mood disorders.

In the clinic, 40-50% of depressed patients who are initially prescribed antidepressant therapy do not experience a timely remission of depression symptoms. This group typifies treatment-refractory depression, that is, a failure to demonstrate an “adequate” response to an “adequate” treatment trial (that is, sufficient intensity of treatment for sufficient duration)). Moreover, about 20-30% of depressed patients remain partially or totally resistant to pharmacological treatment

This is increasing evidence implicating the role of neurotransmitters in depression, in particular the monoamines serotonin, noradrenaline, dopamine, as well as the excitatory amino acid glutamate. Many of the tricylic antidepressants (TCAs), selective serotonin re-uptake inhibitors (SSRIs) and monoamine oxidase inhibitors (MAOIs) effective in the treatment of depression increase the availability of the catecholamines (noradrenaline and dopamine) and indolamines (serotonin) in the central nervous system (CNS). The clinical efficacy of these agents has given rise to the catecholamine-indolamine hypothesis of depression. This theory postulates that a certain level of amines and/or receptor sensitivity to catecholamines functions to generate a normal mood. Receptor insensitivity, a depletion of monoamines, or a decrease in their release, synthesis or storage have been postulated to lead to depression.

Although previous work has suggested the use of certain SNPs to diagnose depression (see, for example, US 2008/0299125 to Hinds et al., US 2008/0199866 to Akil et al., US 2008/0268436 to Duan et al., US 2006/0160119 to Turner et al., US 2008/018076 to Chissoe, and US 2008/0118918 to Licinio et al.), the systems, assays and methods described herein are based on the discovery that the behavioral phenotypes of gene expression can be understood and interpreted in terms of the net effect of these particular genes on either excitatory or inhibitory networks in brain synaptic pathways. Thus, as described herein, by determining a patient's genetic disposition based specifically on the contribution of SNPS that enhance or inhibit the expression or activity of CaMKII, the net effect on CaMKII can be determined and used to guide treatment in ways that was not possible or suggested by previous work.

We herein propose that antidepressant drug action is the result of adaptive changes in neuronal signaling mechanisms, rather than a primary effect on neurotransmitter transporters, receptors, or metabolic enzymes. Among the signaling mechanisms involved, presynaptic signaling (calcium/calmodulin-dependent protein kinase II [CaMKII]) plays a critical role in modulating antidepressant therapy. Modulation of the presynaptic signaling based on knowledge of all (or an appropriate subset) of the genetic factors, including SNPs, influencing the presyaptic signaling may provide improvements over currently applied treatments that do not take into account the net influence of these factors.

### SUMMARY OF THE INVENTION

We herein postulate herein that depression subtypes are based upon imbalances between excitatory and inhibitory mechanisms in the brain. Certain subtypes of depression are associated with predominant excitatory pathways which involve abnormal expression of genes and neurotransmitters leading to specific phenomenological behavioral states. In other, distinct forms of depression, inhibitory pathways predominate, with abnormal expression of a separate and distinct set of genes and neurotransmitters. Thus, a clinician will be able to ascertain a specific subtype of depression by analyzing both the behavioral and genetic patterns of individuals with a mood disorder.

In particular, we propose herein that changes in synaptic plasticity are involved in the pathophysiology of depression and are also involved in the mechanism of antidepressants. In particular, understanding the balance of activity of Ca2+/calmodulin-dependent protein kinase II (CaMKII) is critical to effective treatment of depression, and potentially other mental disorders.

As used herein, the term “mood disorder” may include any number of disorders, including, but not limited to: major depression, bipolar disease, psychotic disorders, childhood disorders, geriatric disorders, anxiety disorders, PTSD, and the like.

CaMKII is markedly enriched at synapses, where it is involved in the control of synaptic transmission, transmitter release and synaptic plasticity. CaMKII is a protein kinase that is involved in synaptic plasticity, has been previously shown to be a target of antidepressants. However, different classes of antidepressants have divergent effects on the expression and function of this kinase, suggesting the involvement of different mechanisms of activation. Inhibitors of CaMKII modulate the stoichiometry of 5HT flux and Serotonin transport activity, resulting in reduced uptake and subsequently higher levels of synaptic serotonin Conversely, other antidepressants appear to have opposite effects on CaMKII. For instance, tianeptine, an atypical antidepressant, stimulates the activity of CaMKII and SERT transport.

This observation may describe a new putative site of action and classification of psychotropic drugs, as well as a previously undisclosed explanation on how single nucleotide polymorphisms (SNPs) in certain genes are related to subtypes of depression. In this model, certain antidepressants and other psychotropic agents mediate their effects via inhibition of CaMKII. Such agents reduce cortical excitability. The decision to employ this class of agents can be assisted by an analysis of gene polymorphisms which are associated with up-regulation of CaMKII.

However, a separate and phenotypically distinct group of patients with neuropsychiatric disorders are characterized by an imbalance in inhibitory neurotransmission. In this subtype, the target of a therapeutic requires activation, not inhibition, of CaMKII. The identification of these individuals can be determined by an analysis of a second, distinct subset of genes which results in reduced CaMKII activity. Subsequently, psychotropic agents which activate CaMKII are preferentially indicated. Thus, described herein are methods, devices and systems (e.g., assays) for determining if a patient has one or more SNPs effecting the expression of genes that either reduce or increase the activity or expression of genes that ultimately modulate the activity of CaMKII. Further, also described herein are methods, devices and systems for providing treatment guidance based on the identification of one or more of these SNPs.

Epistasis refers to the phenomenon where the effects of one gene are modified by one or several other genes, which are sometimes called modifier genes. The gene whose phenotype is expressed is said to be epistatic.

As mentioned, CaMKII is markedly enriched at synapses, where it is involved in the control of synaptic transmission, transmitter release and synaptic plasticity. We herein propose that alterations of the activity of CaMKII form the basis of gene, environment and drug related effects on behavioral states.

Communication between cell surface proteins and the nucleus is integral to many cellular adaptations. In the case of ion channels in excitable cells, the dynamics of signaling to the nucleus are particularly important because the natural stimulus, surface membrane depolarization, is rapidly pulsatile. CaMKII acting near the channel couples local Ca(2+) rises to signal transduction, encodes the frequency of Ca(2+) channel openings, and amplifies molecular signals in the brain

Calcineurin is a calmodulin (CaM) dependent protein phosphatase recently found to be altered in the brains of patients suffering from schizophrenia and by repeated antipsychotic treatment. Repeated treatment with haloperidol, clozapine or risperidone decrease CaMKIIalpha, whereas increases in this protein were observed in an amphetamine model of the positive symptoms of schizophrenia.

Lithium is widely used in the treatment of bipolar disorder, although its mechanism of action is not fully clear. Lithium down-regulates CaMKIV (enzymatic activity, phospho-Thr196 and protein expression level) in the hippocampus, indicating the involvement of CaMKIV in the mechanism of action of lithium.

Intracellular calcium influx through NMDA receptors triggers a cascade of deleterious signaling events which lead to neuronal death Inhibitors of Ca(2+)/calmodulin-dependent protein kinase II (CaMKII) prevent the occurrence of apoptosis, suggesting a role for CaMKII in NMDA mediated cell death.

D₁ receptor (Dopamine) pathways in the prefrontal cortex are linked to working memory and behavior and have been shown to modulate long-term potentiation of intrinsic excitability through the activation of CaMK pathways. Evidence for a role of CaMK pathways in PFC-dependent processes and its connections with COMT demonstrate increased levels of CaMK with lower COMT enzyme activity and higher prefrontal dopamine. Thus, it may be predicted that in genetic polymorphisms of COMT in which there is less enzymatic activity, higher prefrontal dopamine is associated with elevated CAMKII.

This observation discloses a new putative site of action and classification of psychotropic drugs, as well as a previously undisclosed explanation on how single nucleotide polymorphisms in various genes are related to subtypes of depression. In this model, certain antidepressants and other psychotropic agents mediate their effects via inhibition of CaMKII. These agents reduce cortical excitability. The decision to employ this class of agents can be assisted by an analysis of gene polymorphisms which are associated with up-regulation of CaMKII.

However, a separate and phenotypically distinct group of patients with neuropsychiatric disorders are characterized by an imbalance in inhibitory neurotransmission. In this subtype, the target of a therapeutic requires activation, not inhibition, of CaMKII. The identification of these individuals can be determined by an analysis of a second, distinct subset of genes which results in reduced CaMKII activity. Subsequently, psychotropic agents which activate CaMKII are preferentially indicated

A cluster of genes currently claimed to be in epistasis in excitatory patterns of depression include: CACNA1C, GRIK4, FKBP5, TREK-1, 5HT1A and the SERT long allele subtype. Specific interventions based upon these gene clusters is also claimed. These treatments promote inhibitory mechanisms in the CNS based upon their specific effects on the abnormal expression of these genes. These agents include kainite receptor antagonists including Riluzole and natural as well as synthetic cannabanoids. In other subtypes of depression which are characterized by HPA axis activation, including FKBP5, CACNA1C and TREK, calcium channel blockers active in the CNS, such as Candesartan, Fasudil and Flunarazine can be used to reduce abnormal excitatory patterns.

A cluster of genes claimed to be in epistasis associated with a primary inhibitory pattern of depression includes: BDNF, COMT val/val subtype and the short allele of the serotonin transporter. Treatments claimed which are specifically designed to up-regulate impaired neurogenesis mechanisms associated with depression are also claimed. These include Tianeptine, Aniracetam and other racetams, including Nefiracetam which act as AMPA receptor agonists. Preferred embodiments include Tianeptine in combination with Aniracetam or other Racetams such as Nefiracetam.

Thus, as described herein, certain subtypes of depression are characterized by up-regulated excitatory pathways. Excitatory pathways may include polymorphisms in the FKBPS, CACNA1C, COMT met/met and TREK pathways. Novel and previously undisclosed treatments to treat subjects demonstrating these polymorphisms are claimed. These include Riluzole in combination with natural or synthetic cannabinoids to treat GRIK4 polymorphisms, and (2) Candesartan, Fasudil or Flunarazine to treat polymorphisms in the FKBP5, TREK 1 and CACNA1C genes. Atypical neuroleptics such as Aripiprazole can be included for individuals with COMT met/met polymorphisms.

In a second depression subtype, polymorphisms in the SERT allele, COMT val/val and BDNF gene are claimed to be in epistasis, resulting in impaired neurogenesis. In this group of mood disorder patients, Tianeptine and AMPAkines such as Aniracetam and Nefiracetam are described.

For example, described herein are panel assays to determine the presence of SNPs that up-regulate or inhibit CaMKII activity. Such panel assays may include: a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis.

The panel assay may also include an interpretive comment indicating the effect of any identified SNPs on the regulation of CaMKII activity. In some variations, the panel assay includes an interpretive comment suggesting a treatment based on identified SNPs.

In general, and SNP indicator indicates the presence or absence of an SNP from a tissue sample. The SNP indicator may be based a screening test, such as a genetic screen (e.g., using a PCR-based test) to determine if the SNP is present within the DNA of a particular patient's tissue sample being examined. Any appropriate test for the individual SNP, or a pooled test for multiple SNPs may be used as part of the methods, kits, assays and systems described herein. As mentioned, the SNP indicators comprise one or more PCR-based assays. An SNP indicator may include a report (e.g., visual, oral, printed, electronic, or the like), and may indicate the presence or absence of the particular SNP. The SNP indicator may indicate if the SNP is homozygous or heterozygous.

For example, in some variations, the SNP indicator indicates an SNP that alters the function or expression of the 5HT1a, SERT and BDNF genes in the serotonin metabolism pathway. In some variations, the SNP indicator indicates an SNP that alters the function or expression of the MTHF, TREK, or COMT genes in the dopamine metabolism pathway. In some variations, the SNP indicator indicates an SNP that alters the function or expression of the CACNA1C or GRIK4 genes in the glutamate metabolism pathway. In some variations, the SNP indicator indicates an SNP that alters the function or expression of the TREK, FKBP5 or CACNA1C genes in the hypothalamic pituitary adrenal axis.

Also described herein are panel assays to determine the presence of SNPs that up-regulate or inhibit CaMKII activity. The panel assay may include: a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and an interpretive comment indicating the effect of any identified SNPs on the regulation of CaMKII activity.

In some variations, the interpretive comment indicates no effect, up-regulation or down-regulation of CaMKII.

As mentioned above, the assay may also include an interpretive comment suggesting a treatment based on identified SNPs.

Also described herein are panel assays to determine the presence of SNPs that up-regulate or inhibit CaMKII activity. For example, a panel assay may include: a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and an interpretive comment suggesting a treatment based on the identified SNPs.

The assay may also include an interpretive comment indicating the effect of any identified SNPs on the regulation of CaMKII activity.

Also described herein are kits to determine the presence of SNPs that up-regulate or inhibit CaMKII activity. Such a kit may include: an SNP assay indicating the presence or absence of an SNP that alters the function or expression of a gene in the serotonin metabolism pathway; an SNP assay indicating the presence or absence of an SNP that alters the function or expression of a gene in the dopamine metabolism pathway; an SNP assay indicating the presence or absence of an SNP that alters the function or expression of a gene in the glutamate metabolism pathway; and an SNP assay indicating the presence or absence of an SNP that alters the function or expression of a gene in and the hypothalamic pituitary adrenal axis.

In some variations of the kit, the SNP assay indicates an SNP that alters the function or expression of the 5HT1a, SERT and BDNF genes in the serotonin metabolism pathway. In some variations, the SNP assay indicates an SNP that alters the function or expression of the MTHF, TREK, or COMT genes in the dopamine metabolism pathway. In some variations, the SNP assay indicates an SNP that alters the function or expression of the CACNA1C or GRIK4 genes in the glutamate metabolism pathway. In some variations, the SNP assay indicates an SNP that alters the function or expression of the TREK, FKBP5 or CACNA1C genes in the hypothalamic pituitary adrenal axis.

The kit may also include interpretive logic configured to analyze the results of all of the SNP assays and to provide an interpretive comment, wherein the interpretive logic is encoded for processing on a processor. The interpretive comment indicates the effect of any identified SNPs on the regulation of CaMKII activity. The interpretive comment suggests a treatment based on the identified SNPs. The interpretive logic may be configured to propose a treatment to inhibit CaMKII when the identified SNPs up-regulate CaMKII, and further wherein the interpretive logic is configured to propose a treatment to preferentially activate CaMKII activity when the identified SNPs down-regulate CaMKII.

Also described herein are methods of determining the presence of SNPs that up-regulate or inhibit CaMKII activity in a subject. These methods may include: assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis. The sample may be any appropriate tissue sample, including but not limited to a blood sample.

The method may also include the step of indicating that CaMKII activity is up-regulated or down-regulated based on the assayed SNPs. The step of assaying may include assaying for an SNP that alters the function or expression of the 5HT1a, SERT and BDNF genes in the serotonin metabolism pathway. The step of assaying may comprise assaying for an SNP that alters the function or expression of the MTHF, TREK, or COMT genes in the dopamine metabolism pathway. The step of assaying may comprise assaying for an SNP that alters the function or expression of the CACNA1C or GRIK4 genes in the glutamate metabolism pathway. The step of assaying may comprise assaying for an SNP that alters the function or expression of the TREK, FKBP5 or CACNA1C genes in the hypothalamic pituitary adrenal axis.

Also described herein are methods of determining a treatment for depression in a subject by determining the presence of SNPs that up-regulate or inhibit CaMKII activity in the subject. The method may include the steps of: assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis; and proposing a treatment for depression based on the presence the SNP detected by assaying the sample.

The step of assaying may comprise assaying for an SNP that alters the function or expression of the 5HT1a, SERT and BDNF genes in the serotonin metabolism pathway. The step of assaying may comprise assaying for an SNP that alters the function or expression of the MTHF, TREK, or COMT genes in the dopamine metabolism pathway. The step of assaying may comprise assaying for an SNP that alters the function or expression of the CACNA1C or GRIK4 genes in the glutamate metabolism pathway. The step of assaying may comprise assaying for an SNP that alters the function or expression of the TREK, FKBP5 or CACNA1C genes in the hypothalamic pituitary adrenal axis.

Also described herein are methods of determining a treatment for depression in a subject by determining the presence of SNPs that up-regulate or inhibit CaMKII activity in the subject. For example, the method may include the steps of: assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis;

determining the net effect of any SNPs detected by assaying the sample on CaMKII activity; and proposing a treatment for depression that inhibits CaMKII activity if the net effect is to up-regulate CaMKII activity, or that preferentially activates CaMKII activity if the net effect down-regulates CaMKII activity.

The step of assaying may comprise assaying for an SNP that alters the function or expression of the 5HT1a, SERT and BDNF genes in the serotonin metabolism pathway.

The step of assaying may comprise assaying for an SNP that alters the function or expression of the MTHF, TREK, or COMT genes in the dopamine metabolism pathway. The step of assaying may comprise assaying for an SNP that alters the function or expression of the CACNA1C or GRIK4 genes in the glutamate metabolism pathway. The step of assaying may comprise assaying for an SNP that alters the function or expression of the TREK, FKBP5 or CACNA1C genes in the hypothalamic pituitary adrenal axis.

### BRIEF DESCRIPTION OF THE DRAWINGS

FIG. 1 is a flowchart illustrating one variation of a method for determining a net or predicted net effect of one or more SNPs on the CaMKII balance in a patient. This method may be used to propose treatments for depression.

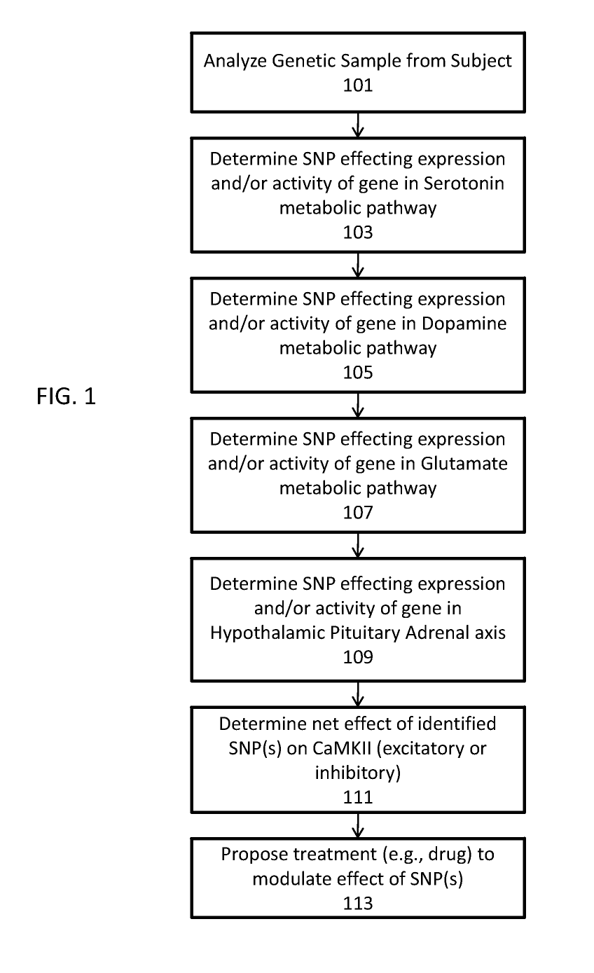

### DETAILED DESCRIPTION OF THE INVENTION

Genes associated with neurotransmitter pathways are abnormal in patients with clinical depression. For instance, genes which regulate serotonin pathways, including genes coding for receptors, metabolism and reuptake mechanisms, are associated with depression. Furthermore, other genetic-neurotransmitter pathways, including dopamine, norepinephrine and glutamate are associated with depression. The heterogenous nature of these results suggests that depression as a disorder is itself heterogenous. By analyzing depression from a single nucleotide polymorphism based gene analysis, subtypes of depression can be differentiated and diagnosed. Accurate subtype depression based upon single nucleotide polymorphism is novel and previously undisclosed. Further, the employment of such analysis will allow mental health professionals who treat individuals with depression with more specific and targeted interventions.

Depression may be better dissected using paradigms that assess how specific genes associate with component features of depression. This approach reveals gene influences on trait components of depression and, may help identify depression subpopulations that can benefit from more targeted pharmacotherapy.

Many people complain of depressive symptoms, but despite the commonality of these complaints, there is significant biochemical heterogeneity regarding the etiology, phenomenology and treatment of mood disorders. This biochemical heterogeneity is evidenced by, inter alia, the occurrence of single nucleotide polymorphisms (SNPs) in genes involved with neurotransmitter activity related to depression. Subtle genomic variations create the chemistry that underlies subtypes of depression.

As an example, a single nucleotide polymorphism in the gene that regulates dopamine can be associated with reduced levels of this neurotransmitter with parallel changes in an individual's behavior. Patients with a dopamine based SNP differ not only in their symptoms but their response to therapies as well.

Based upon the recognition of SNP associated biochemical and symptomatic heterogeneity, a mood complaint such as depression can either be a consequence of a genetic defect that effects serotonin metabolism, but also can be a consequence of an SNP associated genetic defect in a gene that regulates dopamine, or some other neurotransmitter. As a similar example, depression can be etiologically associated with a SNP in glutamate in one individual, and with a SNP related to dopamine or norepinephrine in another.

The recognition of the distinction in the genetic and biochemical heterogeneity related to the expression of subtypes of depression has important therapeutic implications. Frequently, an individual with a mood disturbance does not respond favorably to a specific class of therapeutic agents but may respond to a different class of therapeutic agents. As an example, an individual who is experiencing depression due to a specific SNP related dopamine metabolism defect will not respond or respond less favorably to a serotonin modulating agent. In clinical practice, this can happen when a psychiatrist treats a patient with depression who possesses a SNP associated with a dopamine related defect with a serotonin modulating drug like sertraline or paroxetine instead of a dopamine modulating drug such as buproprion. In these instances, the drug may produce a worsening of symptoms instead of improving them.

Conversely, an individual with a SNP associated with serotonin metabolism will respond less favorably to a dopamine modulating agent. Frequently in such patients, depressive symptoms will not improve or may in fact, worsen. Unfortunately, psychiatrists administer medications for depression solely on a trial and error basis. The lack of diagnostic specificity frequently leads to ineffective treatments or a delay in the proper treatment.

Thus, a common problem in the management of mood disorders is a lack of diagnostic specificity and/or treatments which are not coupled to the unique neurotransmitter disturbance related to depression. Provided herein is a method of using the analysis of an individual's SNPs related to neurotransmitter function as an aid to diagnosis and choice of therapeutic treatment. It is an object of this description to set forth the specific genomic sites that are causally associated with the biochemical and neurochemical abnormalities associated with depression. The ability to accurately identify SNPS related to neurotransmitter imbalances and depression subtypes represents an advance in the field of mental health.

The methods regarding the employment of genes related to neurochemical imbalances are broadly applied to the genes involved in at least the following pathways: Serotonin, dopamine, glutamate and the hypothalamic pituitary adrenal axis. Specific genes within these categories are described in the paragraphs herein but are not limited to this disclosure. Thus, while the present invention describes polymorphisms in specific serotonin pathways, it is recognized that other polymorphisms in the serotonin pathway are contemplated as within the scope of this disclosure.

Genomic polymorphisms in the following glutamate related pathways are associated with depression: CACNAC gene and the kainate receptor gene, referred to herein as GRIK4.

Genomic polymorphisms in the following dopamine related pathways are associated with depression: MTHR gene and COMT gene.

Genomic polymorphisms in the following serotonin related pathways are associated with depression: the serotonin transporter gene and the BDNF gene.

The hypothalamic pituitary adrenal axis has been recognized as a critical region in the stress response as well as in the pathophysiology of depression.

The FKBP5 gene related to the stress response, and the hypothalamic pituitary adrenal pathway, TREK polymorphisms, and the CACNA1C gene polymorphism are currently proposed to be in epistasis as contributing genetic vulnerabilities in depression, based upon their effects on activation of the HPA axis.

These gene categories are associated with depression subendophenotypes, the analysis of which through single nucleotide polymorphisms is applied to provide a more accurate and specific therapeutic intervention based upon the neurochemical consequences of these genetic polymorphisms.

An overriding principal in this claim is that depressed subtypes of mood disorders are due to imbalances of inhibitory and excitatory mechanisms in the brain. The balance between excitatory and inhibitory mechanisms may fundamentally be due to alterations in calmodulin/calcium kinases. In excitatory mood states, excess CAMKII activity results from genetic polymorphisms in TREK, CACNA1c or FKBP5 (via calcineurin).

In depression associated with an imbalance in inhibitory states, such as those associated with reduced BDNF, CAMKII kinase levels are repressed.

A summary of the neurochemical assessment based upon the analysis of single nucleotide polymorphisms is subsequently provided below.

### Glutamate Pathway Associated Genes

GRIK4

The past decade has seen a steady accumulation of evidence supporting a role for the excitatory amino acid (EAA) neurotransmitter, glutamate, and its receptors in depression and antidepressant activity. To date, evidence has emerged indicating that N-methyl-d-aspartate (NMDA) receptor antagonists, group I metabotropic glutamate receptor (mGluR1 and mGluR5) antagonists, as well as positive modulators of alpha-amino-3-hydroxy-5-methyl-4-isoxazolepropionic acid (AMPA) receptors have antidepressant-like activity in a variety of preclinical models. Moreover, antidepressant-like activity can be produced not only by drugs modulating the glutamatergic synapse, but also by agents that affect subcellular signaling systems linked to EAA receptors (e.g., nitric oxide synthase). In view of the extensive colocalization of EAA and monoamine markers in nuclei such as the locus coeruleus and dorsal raphe, it is likely that an intimate relationship exists between regulation of monoaminergic and EAA neurotransmission and antidepressant effects. Further, there is also evidence implicating disturbances in glutamate metabolism, NMDA, and mGluR receptors in depression and suicidality.

Glutamate is the major fast excitatory amino acid transmitter in the CNS, and exerts its action through receptors that function as ion channels such as the Kainate receptor, which is particularly expressed in the hippocampal formation, have been linked to mood disorders and reduced response to conventional antidepressants.

Recent data from human studies have also highlighted a role for Kainate receptors in certain psychiatric diseases, such as schizophrenia and major depression, and a recent association of Kainate gene variants (GRIK4) with response to antidepressants.

Excitatory glutamate based neurotransmitters interact with CaMKII. Calcium/calmodulin (Ca2+/CaM)-dependent protein kinase II (CaMKII) couples increases in cellular Ca2+ to fundamental responses in excitable cells. Thus, polymorphisms in both the kainite receptor and calcium channels are linked to altered excitatory neurotransmission in the brain and subtypes of depression. Single nucleotide polymorphisms (rs1954787) in the GRIK4 gene, which codes for the kainic acid-type glutamate receptor KA1, in homozygote carriers of the treatment-response-associated marker alleles of GRIK4 were 23% less likely to experience non-response to treatment relative to participants who did not carry this marker allele. Thus, in certain forms of treatment resistant depression, abnormal expression of excitatory kainite receptors are expressed and therapeutic modalities which are specifically designed to attenuate these kainite pathways are required.

The combination of Riluzole and Cannabinoid agents are claimed as a novel and previously undisclosed use for neurological and psychiatric disease states characterized by excess kainite receptor activity. Although this disclosure pertains to neuropsychiatric diseases associated with grik4 polymorphisms, other kainite excess states which require down-regulation of this receptor are also anticipated.

Therapeutic agents claimed to treat depression based upon abnormal genetic expression of the GRIK4 polymorphism include Rilutek and Cannabinoid agents.

Riluzole has neuroprotective activity based upon its ability to inhibit kainite receptors. Exposure to kainate, an L-glutamate analogue, markedly elevates Ca2+ influx and cytosolic [Ca2+], and results in neuronal cell death. Kainate-induced Ca2+ influx and excitotoxicity are blocked by Riluzole. Riluzole, a drug approved for use in the treatment of ALS, produces a potent blockade of presynaptic sodium channels, NMDA, and kainate receptors via a noncompetitive mechanism of inhibition. The amplitude of the responses induced by kainate is significantly reduced in the presence riluzole.

In an open-label study, riluzole was associated with antidepressant effects in individuals with treatment-resistant major depression. The response and remission rates in the present study are comparable to those of other antidepressants in studies of treatment-resistant depression. Thus, while Rilutek has been a suggested therapeutic agent in patients with treatment resistant depression, its specific use in depression associated with GRIK4 polymorphisms has been previously undisclosed.

Doses of Riluzole: The dose(s) of Riluzole depends on the desired effect, on the duration of the treatment, and on the route of administration used. In one embodiment, the dose ranges from 50 mg and 400 mg per day by the oral route for an adult, with unit doses ranging from 25 mg to 200 mg of active substance.

The endogenous cannabinoid system tightly controls neuronal excitability and regulates neuronal excitability via inhibition of kainite receptor activity. Cannabinoids directly target hippocampal glutamatergic neurons to provide protection against acute epileptiform seizures Functional CB1 cannabinoid receptors are present on glutamatergic terminals of the hippocampal formation, colocalizing with vesicular glutamate transporter 1 (VGluT1). The direct endocannabinoid-mediated control of hippocampal glutamatergic neurotransmission may constitute a promising therapeutic target for the treatment of disorders associated with excessive excitatory neuronal activity.

Cannabinoids have been shown to possess anticonvulsant properties via cannabinoid receptor inhibition on kainic acid (KA)-induced epileptiform neuronal excitability. Injection of the high-affinity cannabinoid agonist (−)-11-hydroxy-8-tetrahydrocannabinol-dimethyl-heptyl (HU210, 100 mug kg (−1), i.p.) following KA markedly reduces the burst frequency (% decrease in burst frequency), demonstrating that cannabinoids exert their antiepileptic effects by impeding pathological synchronization of neuronal networks in the hippocampus.

Currently available antidepressant therapies have limited efficacies; consequently, research on new drugs for the treatment of mood disorders has become increasingly critical. Preclinical evidences that cannabinoid agonists can impact mood regulation have opened a new line of research in antidepressant drug discovery, indicating the antidepressant potential of cannabinoid agonists and endocannabinoid enhancers in comparison to standard antidepressants. Fluoxetine increases CB(1) receptor density in the prefrontal cortex. In particular, all antidepressants increase the neurotransmission of serotonin after long-term treatment, enhance the tonic activity of hippocampal 5-HT(1A) receptors, promote neurogenesis, and modulate (decrease or increase) the firing activity of noradrenergic neurons. Interestingly, cannabinoid agonists increase serotonin neuronal firing activity, increase serotonin release in the hippocampus, as well as promote neurogenesis.

Cannabidiol (CBD) is a non-psychotomimetic constituent of Cannabis sativa plant that induces anxiolytic effects. The neuroanatomical substrates underlying the effects of THC are linked to depressed activity of CAMKII.

Clinically, open label clinical trials designed to evaluate the effects of nabilone, an endocannabinoid receptor agonist, on treatment-resistant nightmares in patients diagnosed with posttraumatic stress disorder experienced either cessation of nightmares or a significant reduction in nightmare intensity. Subjective improvement in sleep time, the quality of sleep, and the reduction of daytime flashbacks and nightsweats were also noted by some patients. The results of this study indicate the potential benefits of nabilone, a synthetic cannabinoid, in patients with PTSD.

The neuropharmacological properties of cannabinoids, including cannabinoids Delta(9)-tetrahydrocannabinol (THC) and cannabidiol (CBD) may exert sedative, hypnotic, anxiolytic, antidepressant, antipsychotic and anticonvulsant effects. Pure synthetic cannabinoids, such as dronabinol and nabilone and specific plant extracts containing THC, CBD, or a mixture of the two in known concentrations, are available and can be delivered orally or sublingually

Formulations containing specific, defined ratios of cannabinoids may be formulated from pure cannabinoids in combination with Riluzole, as the combination has synergistic effects on inhibiting the kainite receptor. Pharmaceutical grade “pure” cannabinoids may be purchased from commercial suppliers, for example CBD and THC can be purchased from Sigma-Aldrich Company Ltd, Fancy Road, Poole Dorset, BH12 4QH, or may be chemically synthesized. Alternatively, cannabinoids may be extracted from Cannabis plants using techniques well-known to those skilled in the art.

In preferred embodiments of the invention the formulations comprise extracts of one or more varieties of whole Cannabis plants, particularly Cannabis sativa, Cannabis indicia or plants which are the result of genetic crosses, or synthetic cannabis derivatives such as Nobilone which are combined with the known kainite antagonist riluzole to treat excess neuronal states associated with psychiatric disorders. These disorders may be diagnosed by methods described in this work, i.e., by the presence of GRIK4 polymorphisms, or may be treated empirically without such tests being available.

CACNA1C (rs1006737) G>A and (rs10848635) T>A

The calcium ion is one of the most versatile, ancient, and universal of biological signaling molecules, known to regulate physiological systems at every level from membrane potential and ion transporters to kinases and transcription factors. Disruptions of intracellular calcium homeostasis underlie a host of emerging diseases, the calciumopathies. Cytosolic calcium signals originate either as extracellular calcium enters through plasma membrane ion channels or from the release of an intracellular store in the endoplasmic reticulum (ER) via inositol triphosphate receptor and ryanodine receptor channels. Therefore, to a large extent, calciumopathies represent a subset of the channelopathies, but include regulatory pathways and the mitochondria, the major intracellular calcium repository that dynamically participates with the ER stores in calcium signaling, thereby integrating cellular energy metabolism into these pathways, a process of emerging importance in the analysis of the neurodegenerative and neuropsychiatric diseases.

Molecular genetic analysis offers opportunities to advance our understanding of the nosological relationship between psychiatric diagnostic categories in general, and the mood and psychotic disorders in particular. The CACNA1C (alpha 1C subunit of the L-type voltage-gated calcium channel; SNP example rs1006737) gene encodes one subunit of a calcium channel. Results suggest that ion channelopathies may be involved in the pathogenesis of bipolar disorder, schizophrenia and autism with an overlap in their pathogenesis based upon disturbances in brain calcium channels.

CACNA1C encodes for the voltage-dependent calcium channel L-type, alpha 1c subunit. Gene variants in CACNA1 are associated with altered calcium gating and excessive neuronal depolarization. CACNA1 polymorphisms such as rs 10848635 and 1006737 are associated with increased risk of bipolar disease, risk of SSRI induced suicidal ideation and changes in baseline agitation. Significant effects have been found of the G to A variant on total gray matter volume.

Psychiatric disease phenotypes, such as schizophrenia, bipolar disease, recurrent depression and autism, produce a constitutionally hyperexcitable neuronal state that is susceptible to periodic decompensations. The gene families and genetic lesions underlying these disorders may converge on CACNA1C, which encodes the voltage gated calcium channel which can be diagnostically evaluated for its role in schizophrenia, autism and bipolar disease.

Recent genetic studies found the A allele of the variant rs1006737 in the alpha 1C subunit of the L-type voltage-gated calcium channel (CACNA1C) gene to be overrepresented in patients suffering from bipolar disorder, schizophrenia or major depression

Strong evidence of association at the polymorphism rs1006737 (within CACNA1C, the gene encoding the alpha-1C subunit of the L-type voltage-gated calcium channel) with the risk of bipolar disorder (BD) has recently been reported in a meta-analysis of three genome-wide association studies of BD, including the BD sample (N=1868) studied within the Wellcome Trust Case Control Consortium. In a UK case sample of recurrent major depression (N=1196) and schizophrenia (N=479) and UK non-psychiatric comparison groups (N=15316) to examine the spectrum of phenotypic effect of the bipolar risk allele at rs1006737, it was found that the risk allele conferred increased risk for schizophrenia and recurrent major depression with similar effect sizes to those previously observed in BD. These findings suggest some degree of overlap in the biological underpinnings of susceptibility to mental illness across the clinical spectrum of mood and psychotic disorders, and show that at least some loci can have a relatively general effect on susceptibility to diagnostic categories based upon alterations in calcium signaling.

Agents claimed as having application to treat neuropsychiatric disorders associated with altered calcium signaling include: Flunarazine, candesartan and Hydroxyfasudil

For example, Hydroxyfasudil may affect a protein kinase that serve to catalyze the phosphorylation of an amino acid side chain in various proteins. These enzymes control the majority of the signaling processes inside cells, thereby governing cell function, growth, differentiation and destruction (apoptosis) through reversible phosphorylation of the hydroxyl groups of serine, threonine and tyrosine residues in proteins.

Accordingly, serine/threonine kinases and the signal transduction pathways which they are part of are important targets for drug design.

A major signal transduction system utilized by cells is the RhoA-signalling pathway. RhoA is a small GTP binding protein that can be activated by several extracellular stimuli such as growth factor, hormones, mechanic stress, or osmotic change as well as high concentration of metabolite like glucose. RhoA activation involves GTP binding, conformation alteration, post-translational modification and activation of its intrinsic GTPase activity. Activated RhoA is capable of interacting with several effector proteins including ROCKs (Rho kinase) and transmit signals into cellular cytoplasm and nucleus. Abnormal activation of the Rho/ROCK pathway has been observed in various disorders

Injury to the brain and spinal cord activates ROCKs, thereby causing neurodegeneration and inhibition of neuroregeneration like neurite growth and sprouting Inhibition of ROCKs results in induction of new axonal growth, axonal rewiring across lesions within the CNS, accelerated regeneration and enhanced functional recovery after acute neuronal injury.

1-(5-Isoquinolinesulfonyl)homopiperazine hydrochloride (hereinafter referred to as “fasudil hydrochloride”) is commercially available under the trademark of “Eril Inj.” (manufactured by Asahi Kasei Pharma Corp.) and clinically used as an injection preparation for improving cerebrovascular spasm after a subarachnoid bleeding operation and an accompanying brain ischemia.

Hydroxy fasudil is a specific Rho-kinase inhibitor which suppresses the increase of [Ca(2+ induced by Glutamate. The neuroprotective effect of hydroxy fasudil is attributed to repressing Glu excitotoxicity and calcium overload by inhibiting Ca(2+) release from Ca(2+) stores in neurons.

The use of fasudil as an orally bioavailble, novel antidepressant based upon its calcium mediated neuronal stabilization effects has been previously undisclosed.

Candesartan has been shown to modify the Angiotensin II response in tissue. Angiotensin II (Ang II) is a powerful signaling molecule in neurons and exerts some of its biological effects by modulating Ca(2+) currents The physiological actions of Ang II in the brain, whether mediated by AT1 or AT2 receptors, involve changes in neuronal activity that are initiated by changes in the activity of membrane ionic currents and channels, intracellular signalling pathways couple neuronal AT1 and AT2 receptors to changes in the activity of membrane K+ and Ca2+ currents and channels.

Intracellular Ca2+ is known to play an important role in Ang II signaling in neurons and Ang II caused a rapid time-dependent increase in [Ca2+]I voltage-sensitive Ca2+ channels, which are the primary source of Ang II-induced increases in [Ca2+].

This observation leads to a previously undisclosed use of Candesartan or other ARB agents to treat neuropsychiatric disorders associated with abnormal calcium signaling in the brain. Candesartan, an AT(1) blocker, can improve conditions associated with abnormal Ca(2+) release mechanisms due to the observation that AT(1) receptor blockade protects neurons of cellular alterations typically associated with calcium mediated hyperexcitability. Therefore, prevention of these alterations by candesartan may present a useful and novel pharmacological strategy for the treatment of neuropsychiatric disorders associated with altered calcium signaling in the brain, such as mood disorders, autism, bipolar disease and schizophrenia

A variety of A-II antagonists are, or will be, known to one skilled in the art. Subcutaneous or oral administration of the ARB candesartan inhibits brain as well as peripheral AT(1) receptors, indicating transport across the blood-brain barrier, making it the preferred embodiment of this invention because this invention applies to a novel use of this agent to treat disorders of the CNS.

Flunarizine is known as a nonspecific calcium channel blocker that has been used for decades for the treatment of migraine, vertigo, and cognitive deficits related to cerebrovascular disorders. Flunarizine also has dopamine D2 receptor blocking properties and was effective in animal models of predictive validity for antipsychotics. However, its clinical antipsychotic efficacy compared to haloperidol demonstrated no significant differences in PANSS overall score. It has a unique pharmacokinetic profile as an oral drug with long half-life (2-7 weeks).

The use of flunarazine in patients with mood disorders associated with cacnalc polymorphisms has been previously undisclosed.

In some variations, a combination of Candesartan with either Flunarazine or Fasudil may be used (or proposed for treatment) for individuals with CACNA1C polymorphisms.

### Stress Response and the Hypothalamic-Pituitary Adrenal Axis Genes in Depression

FKBP5

FKBP5 regulates the cortisol-binding affinity and nuclear translocation of the glucocorticoid receptor. FKBP5 is a glucocorticoid receptor-regulating co-chaperone of hsp-90 and plays a role in the regulation of the hypothalamic-pituitary-adrenocortical system and the pathophysiology of depression.

FK506 regulates glucocorticoid receptor (GR) sensitivity. When it is bound to the FKBP5 receptor complex, cortisol binds with lower affinity and nuclear translocation of the receptor is less efficient. FKBP5 expression is induced by glucocorticoid receptor activation, which provides an ultra-short feedback loop for GR-sensitivity.

Changes in the hypothalamic-pituitary-adrenocortical (HPA) system are characteristic of depression. Because the effects of glucocorticoids are mediated by the glucocorticoid receptor (GR), and GR function is impaired in major depression, due to reduced GR-mediated negative feedback on the HPA axis. Antidepressants have direct effects on the GR, leading to enhanced GR function and increased GR expression.

Polymorphisms the gene encoding this co-chaperone have been shown to associate with differential up-regulation of FKBP5 following GR activation and differences in GR sensitivity and stress hormone system regulation. Alleles associated with enhanced expression of FKBP5 following GR activation, lead to an increased GR resistance and decreased efficiency of the negative feedback of the stress hormone axis. This results in a prolongation of stress hormone system activation following exposure to stress. This dysregulated stress response might be a risk factor for stress-related psychiatric disorders.

Various studies have identified single nucleotide polymorphisms (SNPs) in the FKBP5 gene associated with response to antidepressants, and one study found an association with diagnosis of depression. Polymorphisms at the FKBP5 locus have also been associated with increased recurrence risk of depressive episodes. A recent study showed that FKBP5 genotypes also moderated the risk of post-traumatic stress disorder (PTSD). Four single-nucleotide polymorphisms (SNPs) in FKBP5, rs3800373, rs9296158, rs1360780, and rs9470080, were genotyped on the complete sample.

In fact, the same alleles are over-represented in individuals with major depression, bipolar disorder and post-traumatic stress disorder.

Individuals homozygous for the TT-genotype at one of the markers (rs1360780) reported more depressive episodes and responded better to antidepressant treatment.

The stress hormone-regulating hypothalamic-pituitary-adrenal (HPA) axis has been implicated in the causality as well as the treatment of depression. recurrence of depressive episodes with single-nucleotide polymorphisms in FKBP5, a glucocorticoid receptor-regulating cochaperone of hsp-90. These single-nucleotide polymorphisms were also associated with increased intracellular FKBP5 protein expression, which triggers adaptive changes in glucocorticoid receptor.

Major depression is associated with reduced hippocampal volume linked to stress and high glucocorticoid secretion.

In animal models, pretreatment with candesartan profoundly modifies the response to stress. The ARB prevents the central sympathetic activation characteristic of isolation stress and abolishes the activation of the hypothalamic-pituitary-adrenal axis during isolation.

Angiotensin II, through AT(1) receptor stimulation, is a major stress hormone, and that ARBs, in addition to their antihypertensive effects, may be considered for the treatment of neuropsychiatric disorders associated with FKBP5 polymorphisms

Long-term pretreatment with an angiotensin II AT1 antagonist blocks angiotensin II effects in brain and abolishes the hypothalamic-pituitary-adrenal responses to isolation stress. AT1 receptor blockade prevented the isolation-induced increase in brain AT1 receptors and decrease in AT2 binding in the locus coeruleus. In addition, pretreatment with candesartan increased the time spent in and the number of entries to open arms of the elevated plus-maze, measure of decreased anxiety.

Calcium/calmodulin (Ca2+/CaM)-dependent protein kinase II (CaMKII) couples increases in cellular Ca2+ to fundamental responses in excitable cells. CaMKII is activated by angiotensin II, providing evidence that calcium signaling in the brain is activated by angiotensin II. The Ang II-induced apoptotic cascade converges in a common pathway mediated by CaMKII activation which results in p38MAPK activation and apoptosis.

Conversely, it follows that Angiotensin II blockade results in attenuated brain calcium signaling. It follows that modulation of abnormal calcium signaling via an AT(1) inhibitor may provide a novel means to treat altered calcium signaling associated with polymorphisms in FKBP5 (as well as TREK and CACNA1C as described herein)

A-II antagonist candesartan: 1-(cyclohexyloxycarbonyloxy)ethyl-2-ethoxy-1-[[2′-(1H-tetrazol-5-yl)biphenyl-4-yl]methyl]benzimidazole-7-carboxylate and the pharmaceutically acceptable salts thereof which are disclosed in U.S. Pat. No. 5,196,444, the disclosure of which is incorporated herein by reference.

The dose administered must be carefully adjusted according to age, weight and condition of the patient, as well as the route of administration, dosage form and regimen and the desired result.

A preferred oral dosage form, such as tablets or capsules, will contain candesartan the ARB inhibitor in an amount of from about 1 to about 500 mg, preferably from about 1 to about 100 mg, and more preferably from about 5 to about 50 mg, alone or with a calcium channel blocker, anti-psychotic or mood stabilizer

Fixed combinations of ARB inhibitor and neuroleptic or antipsychotic are more convenient and are preferred, especially in tablet or capsule form for oral administration.

In a preferred embodiment, Candesartan is used with either Fasudil or Flunarazine.

### TREK Polymorphisms

Recently, a role for TREK-1 K⁺ channels in clinical depression has emerged when it was observed that TREK-1 knockout mice displayed a remarkable depression-resistant phenotype.

Background potassium channels determine membrane potential and serve as prominent effectors for modulatory regulation of cellular excitability in neurons. TREK-1 is a two-pore domain background K⁺ channel (KCNK2) that regulates ACTH and cortisol secretion.

Stress activates the hypothalamic-pituitary-adrenal (HPA) axis, releasing ACTH from the anterior pituitary gland and glucocorticoids from the adrenal cortex. TREK channels are implicated in regulation of cellular excitability in these systems. TREK-1 functions pivotally in the physiology of adrenocorticotropic hormone- and angiotensin II-stimulated cortisol and aldosterone secretion.

Recent findings indicate that genetic variation in KCNK2 may identify individuals at risk for treatment resistant depression. Deletion of the two-pore domain potassium channel TREK-1 results in an antidepressant-like phenotype. TREK-1-deficient mice behave as if they have been treated with an antidepressant drug, such as fluoxetine. Moreover, TREK-1-deficient mice showed a reduced elevation of corticosterone level under stress. These channels regulate the neural circuitry and endocrine output of the HPA axis and indicate that the stress hyporesponsiveness in TREK null mice results from reduced activation of hypothalamic neurosecretory neurons.

Genetic analysis of patients with treatment resistant depression exhibit polymorphism in TREK genes may include the following Single Nucleotide Polymorphisms (SNPs): rs 6686529, 6686521, 2841616, 2841608

KCNK2 (rs6686529) C>G; (rs2841616) A>G; (rs2841608) A>C

KCNK2 gene variants have been associated with resistance to multiple classes of antidepressants.

rs6686529 GG homozygotes show greater susceptibility to MDD than CG heterozygotes; CC carriers, specifically, have a greater possibility of achieving remission after 8 weeks of therapy than G-allele carriers.

rs2841616 and rs2841608 are associated with “Level 2” remission (STAR-D). rs3841616 is also associated with “Level 3” treatment with unsatisfactory benefit to citalopram and second-step pharmacotherapy.

These findings suggest the importance of utilizing TREK polymorphism analysis to identify patients at higher risk of treatment resistant depression and also suggest the use of novel antidepressants which act on TREK.

TREK-1 K⁺ channels display a unique pharmacological profile. several agents that potently and preferentially inhibit low-voltage-activated T-type Ca²⁺ channels are also potent TREK-1 antagonists. Specifically, antipsychotics, such as pimozide, inhibit native bTREK-1 channels.

TREK inhibitors are claimed as selective agents for TREK polymorphisms associated with depressive subtypes.

As Trek is a regulator of the HPA axis, it may be in epistasis with both FKBP5 and CACNA1C polymorphisms. Agents claimed as antidepressants based upon these polymorphisms include the calcium channel inhibitors as described in the paragraphs herein, Fasudil and Candesartan.

A combination of Candesartan and Fasudil or Flunarazine is the preferred embodiment for individuals who display FKBP5, CACNA1C or TREK polymorphisms associated with mood disorders.

### Serotonin Associated Genes and BDNF

Serotonin neurotransmitter transporters are the targets of various therapeutic agents used in the treatment of depression and anxiety. Specifically, the selective serotonin reuptake inhibitors, are the most widely prescribed agents for depression. The SSRI mechanism of action in depression is mediated by these agents acting as selective antagonists of the serotonin neurotransmitter transporter. Antagonists block uptake and prolong and/or enhance the action of serotonin SSRI agents, drugs most widely used in depression, selectively block the reuptake of serotonin and result in increased serotonin in the synapse.

### SLC6A4 5-HTTLPR(5-Hydroxytryptamine Transporter Linked Polymorphic Region)

The serotonin transporter (5-HTT) is a high affinity carrier protein, localized to the plasma membrane of the presynaptic neuron. The role of 5-HTT is to remove serotonin (5-HT) from the synaptic cleft, resulting in serotonin reuptake into the presynaptic terminus. Elevated synaptic serotonin levels are associated with improved mood; thus the effectiveness of many antidepressant drugs (namely selective serotonin reuptake inhibitors, SSRIs) is thought to be due to their inhibition of the serotonin transporter, thereby reducing serotonin reuptake into the presynaptic terminus, and increasing serotonin availability in the synaptic cleft. In addition to mood improvement, elevated synaptic serotonin levels are also indirectly associated with a number of negative side effects including sleep disturbances, arousal, decreased gut motility, and sexual dysfunction.

### 5-HTTLPR Polymorphism

The short (S) allele results in 50% less expression of the active transporter protein as compared to the long (L) form. As these genetic differences in the 5-HTT affect both baseline serotonin levels and the availability of the transporter as a target for antidepressant therapy, they can affect the efficacy of antidepressant therapy, the likelihood of side effects, and the nature and extent of depressive symptoms experienced. Studies have shown that compared to L/L patients, those homozygous for the short allele (S/S) are more likely to

a) respond to antidepressant therapy more slowly,

b) experience adverse drug reactions (ADRs) during antidepressant therapy, and

c) develop major depression following adversity due to a poorer stress response.

In general, L/L individuals report a better and faster response to SSRI therapy than S/S patients. While these L/L individuals may demonstrate appropriate response to SSRI therapy in 2 to 4 weeks, individuals with the short allele (L/S or S/S) may respond to SSRI therapy much more slowly or may benefit from non-selective antidepressants.

In a meta analysis regarding the relationship of the serotonin transporter and depression, The SS genotype was significantly associated with an increased risk of MDD among Caucasian populations.

In addition to serotonin transporters being targets for anti depressant therapy, it is also recognized that assessment of serotonin transporter activity may be a useful biomarker in psychiatry. Various studies have demonstrated that patients with serotonin transporter short alleles are less likely to respond to SSRI therapy and are also more likely to experience treatment emergent side effects. The specific gene which is tested for, referred to as either the 5HTTLPR or SLC6A4, regulates the rate of serotonin metabolism. This gene controls a receptor located in the synaptic cleft. The receptor binds to serotonin and shuttles it back to the presynaptic neuron, terminating its activity at the post synaptic junction. The binding affinity of this receptor (referred to as SERT) is regulated by hereditary factors related to the length of an allele. Short alleles have reduced binding affinity effects on the serotonin transporter. Conversely, long alleles have better affinity, resulting in a more efficient reuptake process. Thus, the inherited short allele of the serotonin transporter results in more synaptic serotonin and the inherited long allele leads to reduced serotonin in the synapse. The neurochemical consequences of possessing short alleles of the serotonin transporter results in increased synaptic serotonin, an effect that should be associated with better outcomes in antidepressant treatment based upon the conventional notion that increased synaptic serotonin is equated with better anti depressant response. However, in many studies the patients who are less likely to respond to serotonin agonist anti depressant therapy are precisely those who have a genetic predisposition to have relatively higher levels of serotonin in the synapse. Thus, results of large scale genomic studies which have correlated a percentage of patients who have depression associated with a short allele (and subsequently higher levels of synaptic serotonin), supports the notion that in a unique and previously unrecognized group of patients, there appears to be unique phenotype of depression characterized by higher, rather than lower, synaptic serotonin. It follows that in this unique subset of patients who are characterized by higher synaptic serotonin, the metabolic target should be to enhance serotonin reuptake as opposed to inhibiting serotonin reuptake.

Tianeptine has been described in French Patent Specification FR 2 104 728 as a new medicament for use in the treatment of psychoneurotic disorders. Furthermore, French Patent Specification FR 2 635 461 describes the use of tianeptine and compounds thereof in the treatment of stress. Tianeptine has a unique mechanism of action which is completely opposite drugs which are currently used for depression. Tianeptine not only activates serotonin reuptake into the synaptic ending but also activates its release from the ending into the synaptic cleft thus accelerating serotonin turnover rate in the synapse, a mechanism which is unique and opposite the majority of anti depressants in clinical use (such as the SSRI agents), which increase, rather than decrease synaptic levels of serotonin.

Tianeptine is a clinically used antidepressant that has drawn much attention, because this compound challenges traditional monoaminergic hypotheses of depression. It is now acknowledged that the antidepressant actions of tianeptine can be attributed to its particular neurobiological properties which are opposite those of traditional antidepressants, such as the SSRI class.

Acute treatment with tianeptine significantly enhances the levels of metabolites of 5-HT and 5-hydroxyindole acetic acid in the brain. In contrast to that found with inhibitors of the uptake of 5-HT such as the SSRIs, treatment with tianeptine markedly enhances the depletion of 5-HT. In vitro measurement of the uptake of 5-HT also confirms that tianeptine exerts opposite effects to those of classical SSRI antidepressants, since the in vivo administration of tianeptine induced a significant increase in the uptake of 5-HT in synapses. The fact that both inhibitors of the uptake of 5-HT (SSRIs) and tianeptine which, in contrast, enhances the in vivo uptake of 5-HT, are both potent and efficacious antidepressants, challenges the current hypothesis that SE reuptake is the exclusive mechanism of antidepressant response and that in subsets of patients, the opposite neurochemical effects—i.e., enhanced serotonin reuptake, may be the preferred mechanism to achieve an antidepressant response.

Described herein are novel methods and means for determining the genotype of the serotonin transport gene in order to selectively prescribe a treatment that is ideally coupled to patients with this specific genomic variation. In particular, described herein are methods for the use of tianeptine, of isomers thereof and of salts thereof, intended for the treatment of a specific subtype of depression associated with the short allele of the serotonin transporter.

In general, described herein are methods of treating depression by determining the genotype of an individual patient's serotonin transporter (SERT), and prescribing a modulator of serotonin re-uptake and/or release based on the particular allele of that individual. The use of a genetic test in which a specific polymorphism of the serotonin transport gene is detected which informs the clinician that a patient likely has higher (rather than conventionally predicted lower) serotonin will subsequently alter the decision to choose a SSRE instead of an SSRI

For example, described herein are methods of treating an individual for depression by determining the individuals genotype for the serotonin transporter, and determining the appropriate prescription for a selective serotonin reuptake enhancer (SSRE) drug based on the genotype. Although the primary a selective serotonin reuptake enhancer (SSRE) drug described at the present time is tianeptine (Stablon, Coaxil, Tatinol), the methods described herein may be used with any appropriate a serotonin reuptake enhancer. Tianeptine is currently used as an antidepressant for the treatment or prophylaxis of depression in specific subtypes of depression. The methods described herein include the treatment of subjects exhibiting a particular genotype of the serotonin transporter with a selectively prescribed SSRE (e.g., tianeptine). Thus, the administration of an SSRE (such as tianeptine) is based upon the genotype. For example, the decision to prescribe and/or the dosage of a SSRE may be based upon the length of the patient's serotonin transporter allele. In some variations, patients with the short allele version of the transporter are selectively prescribed tianeptine. An alternative or supplemental treatment may be indicated in patents with longer alleles. For example, patient' with longer alleles may be prescribed serotonin reuptake inhibitors (e.g., SSRIs or other tricyclinc compounds).

Thus, in the above example, a drug such as Tianeptine, which acts specifically as a serotonin reuptake enhancer, would be more appropriate because the metabolic and inherited state of the patient identifies a hyperserotonin state associated with depression.

The methods described herein are based on the recognition that by assessing genotypes (long vs. short alleles) of a polymorphism of the promoter region of the gene that encodes the serotonin transporter (5HTTLPR), one can identify persons who are more likely to respond to alternative anti-depressant therapies based upon unique and seemingly paradoxical effects on serotonin transporter. In these so-identified patients, a novel and previously undisclosed method of use for tianeptine is established based upon the expression and determination of the serotonin transport subtype. Thus, the methods described herein generally include the step of screening subjects for serotonin allele length, which may comprise determining the serotonin transporter gene promoter genotype (with respect to long and short alleles thereof) of a subject. The serotonin transporter gene promoter genotype may be used to indicate whether or not the subject will respond selectively to either a serotonin reuptake inhibitor, or more particularly, a serotonin reuptake agonist.

The methods described herein are particularly adapted to screening for tianeptine responsiveness based upon the expression of single nucleotide polymorphisms in the serotonin transporter. This invention discloses a novel indication for the use of tianeptine based upon the short allele of the serotonin transporter, and a mechanism intended to reduce, rather than enhance, synaptic serotonin.

In one particular embodiment, the method comprises determining the presence of two serotonin transporter gene promoter short alleles in a subject. If a subject is determined to have a depressed subtype characterized by higher synaptic serotonin (secondary to possession of the short allele of the serotonin transporter), tianeptine and/or enantiomers thereof is selectively prescribed, optionally in the form of pharmaceutically acceptable salts, shall be presented in pharmaceutical forms.

In addition to the dosage calibration by genotype, the dosage of the SSRE may vary according to the age and weight of the patient, the administration route, and the nature of the therapeutic indication and associated treatments. For example, in patients for whom tianeptine is indicated based on the genotype, the dose may range from 12.5 mg to 300 mg per dose or per administration. The number of administrations may also be modulated (e.g., 1×, 2×, 3×, 4× per day).

Any appropriate form of the SSRE may be used. For example, regarding tianeptine, bases that convert tianeptine or enantiomers thereof into salts may be used. The preferred salt of tianeptine is the sodium salt.

In some variations, an immediate-release form of the SSRE may be used. Immediate release may lead, in some subjects, to high blood peaks being obtained. A prolonged-release form may also be used. The prolonged-release form may make it possible to avoid these blood peaks and to obtain a uniform blood concentration in man. This may make it possible to reduce undesirable effects which may potentially occur by the “peak effect.” In one variations, a prolonged-release form of the sodium salt of tianeptine may be used to achieve a better therapeutic index in the treatment of anxiety and depression.

The dosage-release for tianeptine may be controlled in any appropriate manner. For example, a matrix tablet (as described in U.S. Pat. No. 5,888,542) that combines a polymer derived from cellulose and a calcium salt, may be used to compound the drug for controlled release of the active ingredient (e.g., tianeptine). This combination may be well-suited to the physicochemical characteristics of the sodium salt of tianeptine.

Controlled release (and particularly near-linear release) may make it possible to obtain a prolonged release of tianeptine leading to blood levels in the range between 50 and 300 ng/ml up to 24 hours after administration of the tablet. As mentioned, in addition to the genotype, the unit dosage may be varied according to the age and the weight of the patient, and the nature and the seriousness of the condition. In general, dosage may range between 12.5 and 50 mg for a daily treatment in patients for whom the genotype screening suggests tianeptine is indicated.

Suitable routes for administration may include oral, parenteral, per- or trans-cutaneous, nasal, rectal, perlingual, sublingual tablets, glossettes, soft gelatin capsules, hard gelatin capsules, lozenges, suppositories, creams, ointments, dermal gels etc., and may include forms allowing the immediate release or the delayed and controlled release of the active ingredient

A method for screening a subject for determining whether said subject is at an increased risk for depressed mood, said method comprising determining the subject's HTTLPR insertion/deletion polymorphism genotype within the serotonin transport (HTT) gene, wherein an LS heterozygote for the HTTLPR insertion/deletion polymorphism in the promoter region of the HTT gene has an increased risk for depressed mood. Subjects having the LS heterozygote for the insertion/deletion polymorphism in the promoter region of the serotonin transporter (HTT) gene have an increased risk of depression.

The short allele of the serotonin transporter has been suggested to be in epistasis with BDNF. For instance, the interaction between 5-HTTLPR and Val66Met polymorphisms significantly predicts dysfunctional thinking from before to after a standardized sad mood provocation. Cognitive reactivity increased among S/L(G) 5-HTTLPR homozygotes if they were also homozygous for the Val Val66Met allele, demonstrating biological epistasis between SLC6A4 and BDNF for predicting connectivity among neural structures involved in emotion regulation.

In order to identify the molecular mechanisms that may contribute to the enhanced susceptibility to depression under serotonin transporter (SERT) dysfunction BDNF levels were significantly reduced in the hippocampus and prefrontal cortex of SERT knockout rats, through transcriptional changes that affect different neurotrophin isoforms. Moreover, BDNF gene expression is also significantly reduced in leukocytes from healthy subjects carrying the S allele of the 5-HTTLPR, suggesting that the changes observed in SERT mutant rats may also be present in humans and may confer enhanced vulnerability to mood disorders.

Some polymorphisms in the promoter region of the serotonin transporter gene (SLC6A4) are also involved in the pathogenesis/treatment of MDD; for instance, a single nucleotide substitution, rs25531 (A/G) in the serotonin transporter is also relevant. A variable number of tandem repeats (short (S) vs long (L)) in the promoter region of the serotonin transporter gene (5-HTTLPR) and a functional variant of a single-nucleotide polymorphism (rs25531) in 5-HTTLPR have been associated with increased risk for major depressive disorder (MDD), this particular variant polymorphism rs 25531, referred to herein as L(g) carriers. In particular, relative to L/L homozygotes, S carriers and L(g)-allele carriers have a higher probability of developing depression after stressful life events. This is because individuals with the rs25531 polymorphism, despite having the long allele of the serotonin transporter, actually behave as if they possess the short allele. Based upon the functional consequences of this SLC6A4 polymorphisms, individuals with the rs25531 are predicted to respond in a similar fashion as those who actually possess the short allele of the transporter with reduced responsive effects to SSRI, more treatment emergent side effects and potentially better response to agents which enhance CaMKII neurogenesis.

BDNF (rs6265) A>G Val66Met

Nefiracetam, Aniracetam and Tianeptine are all agents that may be used to treat patients expressing the BDNF (rs6265) A>G Val66Met SNP.

Brain-derived neurotrophic factor is a member of the nerve growth factor family. It is induced by cortical neurons and is necessary neurogenesis and neuronal plasticity. BDNF has been shown to mediate the effects of repeated stress exposure and long term antidepressant treatment on neurogenesis and neuronal survival within the hippocampus. The BDNF Val66Met variant is associated with hippocampal dysfunction, anxiety, and depressive traits. Previous genetic work has identified a potential association between a Val66Met polymorphism in the BDNF gene and bipolar disorder. Meta-analysis based on all original published association studies between the Val66Met polymorphism and bipolar disorder up to May 2007 shows modest but statistically significant evidence for the association between the Val66Met polymorphism and bipolar disorder from 14 studies consisting of 4248 cases, 7080 control subjects and 858 nuclear families.

The BDNF gene may play a role in the regulation of stress response and in the biology of depression and the expression of brain-derived neurotrophic factor (BDNF) may be a downstream target of various antidepressants.

Exposure to stress causes dysfunctions in circuits connecting hippocampus and prefrontal cortex. BDNF is down-regulated after stress. Acute treatment with the antidepressants tianeptine reverses stress-induced down-regulation of BDNF. Tianeptine, increases the phosphorylation of Ser831-GluA1. Psychological stress down-regulates a putative BDNF signaling cascade in the frontal cortex in a manner that is reversible by the antidepressant tianeptine. Thus agents which promote BDNF are novel mechanisms to treat stress induced alterations in the limbic system

Activation of AMPA receptors by agonists is thought to lead to a conformational change in the receptor causing rapid opening of the ion channel, which stimulates the phosphorylation of CAMK11/PKC sites and subsequently enhance BDNF expression

A structural class of AMPA receptor positive modulators derived from aniracetam are called Ampakines Aniracetam and Nefiracetam are neurological agents called ‘racetams’ that are analogs of piracetam. They are regarded as AMPA receptor potentiators and CaMKII agonists.

Small molecules that potentiate AMPA receptor show promise in the treatment of depression, a mechanism which also appears to be mediated by promoting BDNF via CaMKII pathways. Depression is associated with abnormal neuronal plasticity. AMPA receptors mediate transmission and plasticity at excitatory synapses in a manner which is positively regulated by phosphorylation at Ser831-GluR1, a CaMKII/PKC site.

Aniracetam [1-(4-methoxybenzoyl)-2-pyrrolidinone] is a AMPA receptor potentiator that preferentially slows AMPA receptor deactivation. AMPA receptor potentiators (ARPs), including aniracetam, exhibit antidepressant-like activity in preclinical tests. Unlike most currently used antidepressants. Interactions of aniracetam with proteins implicated in AMPA receptor trafficking and with scaffolding proteins appear to account for the enhanced membrane expression of AMPA receptors in the hippocampus after antidepressant treatment. The signal transduction and molecular mechanisms underlying alpha-amino-3-hydroxy-5-methyl-4-isoxazole propionate (AMPA)-mediated neuroprotection evoke an accumulation of brain-derived neurotropic factor (BDNF) and enhance TrkB-tyrosine phosphorylation following the release of BDNF. AMPA also activate the downstream target of the phosphatidylinositol 3-kinase (PI3-K) pathway, Akt. The increase in BDNF gene expression appeared to be the downstream target of the PI3-K-dependent by AMPA agonists and Tianeptine (described below). Thus, AMPA receptors protect neurons through a mechanism involving BDNF release, TrkB receptor activation, and up-regulation of CaMKII which increase BDNF expression

Olfactory bulbectomized (OBX) mice exhibit depressive-like behaviors. chronic administration (1 mg/kg/day) of nefiracetam, a prototype cognitive enhancer, significantly improves depressive-like behaviors. Decreased calcium/calmoculin-dependent protein kinase II mediates the impairment of hippocampal long-term potentiation in the olfactory bulbectomized mice. Nefiracetam treatment (1 mg/kg/day) significantly elevated CaMKII in the amygdala, prefrontal cortex and hippocampal CA1 regions. Thus, CaMKII, activation mediated by nefiracetam treatment elicits an anti-depressive and cognition-enhancing.

Recommended aniracetam dosage is usually 1500 mg per day, taken in two 750 mg doses, one in the morning and one in the afternoon. Dose ranges can vary between 100 mg-5 grams. Recommended doses of Nefiracetam are 50-200 mg/day

Tianeptine is claimed as an agent to treat depression associated with BDNF. The therapeutic potential of positive AMPA receptor modulators in the treatment of neurological and psychiatric diseases has been previously described, but its use in combination with Tianeptine, an atypical antidepressant with a similar mechanism of action, has been previously undisclosed.

Tianeptine increases BDNF expression in the amygdala, increases in neurotrophic factor expression that may participate in the enhancement of amygdala synaptic plasticity mediated by tianeptine. Preferred embodiments may include Tianeptine with Nefiracetam or Aniracetam in individuals with BDNF polymorphisms, associated with or without SERT ss allele subtype.

BDNF binds to and activates tyrosine kinases receptor (TrkB) to exert its effects. TrkB, after activation by sigma receptor ligands, stimulates phosphoinositide 3-kinase (PI3K). The downstream target of PI3K is Akt-1, which is up regulated by the sigma-1 receptors. The sigma-1 receptor agonist dehydroepiandrosterone (DHEA)-sulfate stimulate increases in the level of Ser473-phosphorylated Akt-1 and that the phosphorylation of Akt-1 mediated by DHEA-s results in a potentiation of BDNF. The use of DHEA-s as an antidepressant selectively in patients who have depression associated with BDNF polymorphisms has not been previously described.

### Dopamine Related Genes

Certain examples pertain to use of the MTHFR gene or related gene products for determining an individuals tendency to experience depression based upon the said individual's inability to methylate certain pathways involved in catecholamine synthesis and or degradation. In one example, diagnosis involves testing a sample obtained from a subject for the presence of a polymorphism in the MTHFR gene.

Certain examples pertain to use of the COMT gene or related gene products for determining an individual's risk of developing or maintaining an addiction based upon the individual's ability to metabolize or maintain normal levels of dopamine in the brain. In one example, diagnosis involves testing a sample obtained from a subject for the presence of a polymorphism in the COMT gene.

The 5,10-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain. Folic acid deficiency results in fatigue, reduced energy and depression. Low folate blood levels are correlated with depression and polymorphisms of the MTHFR gene are closely associated with risk of depression.

MTHFR irreversibly reduces 5-Methyltetrahydrofolate which is used to convert homocysteine to methionine by the enzyme methione synthetase. The c677T SNP of MTHFR has been associated with increased vulnerability to several conditions and symptoms including depression. The neuropsychiatric symptoms associated with c677T genotype occur because MTHF regulates dopamine synthesis. A diminished bioavailability of MTHF as a consequence of a MTHFR polymorphism leads to reduced dopamine synthesis and clinically a distinct subtype of depression is observed.

Nucleotide 677 in the MTHFR gene has two possibilities: C or T. 677C (leading to an alanine at amino acid 222); 677T (leading to a valine substitution at amino acid 222) encodes a thermolabile enzyme with reduced activity. The degree of enzyme thermolability (assessed as residual activity after heat inactivation) is much greater in 677TT individuals (18-22%) compared with 677CT (56%) and 677CC (66-67%)

Suitable MTHF gene polymorphisms include polymorphisms in the 5,10-methylenetetrahydrofolate reductase (MTHFR) gene, including MTHFR C677T and its association with common psychiatric symptoms including fatigue and depressed mood. For unipolar depression, the MTHFR C677T polymorphism has been well described and validated.

COMT is an enzyme involved in the degradation of dopamine, predominantly in the frontal cortex. Several polymorphisms in the COMT gene have been associated with poor cognition, diminished working memory, and increased anxiety as a consequence of altered dopamine catabolism. Suitable COMT gene polymorphisms include, e.g., a polymorphism in a Catechol O-methyltransferase (COMT) gene, the major enzyme determining prefrontal dopamine levels, which has a common functional polymorphism (val(158)met) that affects prefrontal function and working memory capacity and has also been associated with anxiety and emotional dysregulation. A single nucleotide polymorphism in the COMT (Val158/108Met) gene affects the concentration of dopamine in the prefrontal cortex.

The COMT 158val/val genotype confers a significant risk of worse response after 4-6 weeks of antidepressant treatment in patients with major depression. There is a negative influence of the higher activity COMT 158val/val genotype on antidepressant treatment response during the first 6 weeks of pharmacological treatment in major depression, possibly conferred by decreased dopamine availability. This finding suggests a potentially beneficial effect of an antidepressive add-on therapy with substances increasing dopamine availability individually tailored according to COMT val158met genotype.

Dopamine agonists which can be selectively employed to individuals with this COMT polymorphism include MAO inhibitors, Buproprion, and Methylfolate

Studies have indicated that individuals with unique polymorphisms in the COMT gene have varying differences in memory and performance studies. For instance, in met/met homozygotes, there is reduced COMT activity and higher frontal dopamine. Individuals with this polymorphism are more apt to be impulsive and hyperactive due to excess brain dopamine levels.

The COMT gene contains a functional polymorphism (Val158Met) that determines high and low activity of this enzyme. Homozygosity for the low-activity (Met) allele is associated with a three- to four fold reduction of COMT enzyme activity compared with homozygotes for the high-activity (Val) variant, resulting in reduced degradation of synaptic catecholamines in individuals with the Met allele.

COMT met/met individuals represent a heritable variation in which there is excess dopamine neurotransmission associated with the met allele of the COMT polymorphism and which results in heightened reactivity and connectivity in corticolimbic circuits. Common symptoms of these individuals include impulsivity, heightened emotional reactivity, risky behavior and addictions and are less likely to respond to SSRI based antidepressants. Agents which can up-regulate COMT and thereby reduce excess dopamine described in this invention include the atypical neuroleptic, aripiprazole

Chronic treatment of antipsychotic drugs can modulate gene expression in the brain, which may underscore their clinical efficacy. Aripiprazole is the first approved antipsychotic drug of the class of dopamine D2 receptor partial agonist, which has been shown to have similar efficacy and favorable side-effects profile compared to other antipsychotic drugs. Differential gene expression induced by chronic treatment of aripiprazole includes the catechol-β-methyltransferase (Comt) and DNA methyltransferase. Thus, based upon this observation, the use of aripiprazole is claimed for individuals with a mood disorder that demonstrate a COMT met/met polymorphism.

Conversely, individuals who express the val/val variant of the COMT polymorphism have a unique set of psychiatric symptoms. These individuals are frequently associated with the MTHF gene polymorphism as described in the paragraphs above. The predominant disturbance in these individuals is related to impaired methylation with subsequently lower brain dopamine levels in the frontal lobes. The molecular consequences in such individuals would be lower CaMKII activity and reduced working memory. Thus, an additional pharmacological intervention in these individuals would be to apply either an AMPA agonist or Tianeptine to increase impaired CaMKII activity.

The detection of these polymorphisms thus becomes critical to a mental health professional in order to decide whether a dopamine enhancing agent (appropriate for a COMT val/val) is indicated or a different pharmacological agent for individuals who express the COMT met/met allele.

### SNP Detection

As an example, a patient visits with a psychiatrist or other mental health worker. After taking a history, the health care worker obtains a small sample of tissue from the mouth and sends it to a specialized lab which is able to analyze the DNA through methods used to those skilled in the art. The lab determines over a brief period of time the results of the DNA test. As one example, the test indicates whether a patient has one of three subtypes related to the gene, referred to as either LL, LS, or SS (long/long long/short, and short/short) Certain individuals will possess two short alleles. This indicates that the serotonin transporter is less efficient with the short allele than the version in the long allele. The value of this result is as an assessment of serotonin synaptic levels, a more specific serotonin modulation drug can be chosen.

Various real-time PCR methods can be used to detect SNPs, including, e.g., Taqman or molecular beacon-based assays (U.S. Pat. Nos. 5,210,015; 5,487,972; and PCT WO 95/13399) are useful to monitor for the presence of absence of a SNP. Many other SNP detection methods are known in the art, including, without limitation, DNA sequencing, sequencing by hybridization, dot blotting, oligonucleotide array (DNA Chip) hybridization analysis.

Applied Biosystems, Inc (Foster City, Calif.) has developed several aspects of SNP genotyping technology. In one well used protocol PCR amplification of a desired SNP region is conducted using targeting primers, including two allele-specific fluorogenic probes, each consisting of a different fluorescent reporter dye and a fluorescent quencher. Prior to PCR, proximity of the quencher to the fluorphore causes fluorescence resonance energy transfer (FRET), reducing the fluorescence from the reporter dye. During PCR, the 5′ nuclease activity of Taq digests the allele-specific probe bound to the region of the SNP, releasing the fluorescent dye from the quencher and allowing generation of a fluorescence signal.

Any tissue sample may be used for genotyping the polymorphisms described in this art, or for determining levels gene products, including but not limited to, blood, saliva, spinal fluid, brain biopsy, cultured cells, stool, urine, or frozen sections taken for histologic purposes. In certain examples, blood is obtained from a subject for assaying with respect to the mentioned polymorphisms. In an example, venous blood is obtained from a subject using standard venipuncture techniques. In another example, a buccal swab can be obtained for analysis.

### EXAMPLES

Sample test results are displayed

Serotonin Neurotransmission

| | |
|---|---|
| Genes Tested | 5HT1a Receptor, Serotonin Transporter |
| Analytical Results | Genomic polymorphisms are noted in the serotonin |
| | transporter with less efficient presynaptic reuptake |
| | resulting in higher tonic synaptic serotonin. |
| Interpretive Comments | Patients with a polymorphism related to the serotonin |
| | transporter have altered serotonin synaptic |
| | neurotransmission related to reduced uptake |
| | mechanisms and are more likely to have increased |
| | baseline synaptic serotonin. |
| | SSRI intervention in these patients are reportedly less |
| | likely to respond to standard SSRI treatment as these |
| | agents increase synaptic serotonin in patients with |
| | baseline elevations in serotonin. This may be related to |
| | higher rates of treatment related side effects. |
| | Acute enhancement of serotonin in depressed patients |
| | with the short allele of the transporter may unfavorably |
| | alter post synaptic serotonin tonicity Enhanced vigilance |
| | when initiating or discontinuing SSRI therapy is |
| | indicated. Tianeptine, or other Selective serotonin |
| | reuptake enhancers may be preferentially considered in |
| | these individuals |

Dopamine Neurotransmission

| | |
|---|---|
| Genes Tested | Catechol Methyl Transferase, MTHF, |
| Analytical Results | Genomic polymorphisms are noted in the MTHFR and |
| | COMT genes. |
| | Patients with these polymorphisms have higher levels of |
| | dopamine degradation in the prefrontal cortex with potential |
| | reduced executive brain function. |
| | Neuropsychiatric symptoms, including reduced executive |
| | brain potential, have been reported in individuals with excess |
| | COMT related to the val- val polymorphism. |
| Interpretive Comments | Patients with polymorphisms in the MTHFR and COMT |
| | genes may preferentially respond to dopamine agonists or |
| | folic acid; however, this has not been conclusively |
| | demonstrated in clinical studies. Preferential agents in |
| | individuals with COMT val/val polymorphisms, |
| | polymorphisms include: Methylfolate |
| | Preferential therapeutic agents in individuals with COMT |
| | met/met polymorphisms include ariprizaole |
| | Genomic polymorphisms of the TREK potassium channel |
| | have been associated with increased vulnerability to |
| | depression. Conversely, TREK inhibition is associated with |
| | reduced vulnerability to depression. Many classes of |
| | antidepressants inhibit TREK. |
| | TREK may be biochemically linked with dopamine receptors |
| | in the nucleus accumbens. Lower synaptic dopamine in these |
| | brain regions associated with a TREK polymorphism may |
| | lead to a phenotype characterized by anhedonia and reduced |
| | motivation. |
| | TREK regulation has also been associated with satiety |
| | mechanisms |
| | Patients with TREK polymorphisms are less likely to achieve |
| | remission with antidepressants and are more likely to be |
| | diagnosed with treatment resistant depression. Augmentation |
| | agents should strongly be considered in these patients. |
| | Antidepressants which are able to inhibit the TREK channel |
| | include Fluoxetine and certain calcium channel blockers |

Glutamate Neurotransmission

| | |
|---|---|
| Genes Tested | , CACNA1C, GRIK4 |
| Analytical Results | Genomic polymorphisms are noted in the CACNA1C gene |
| | indicating potentially excess neuronal excitability, calcium |
| | ion channel disturbances and excess glutamate. |
| | Alterations in voltage gated calcium ion channels may lead to |
| | abnormal depolarization of selective limbic regions associated |
| | with mood and perception. |
| | CACNA1 polymorphisms have been associated with bipolar |
| | disease, schizophrenia, and treatment resistant depression |
| Interpretive Comments | Patients with CACNA1C polymorphisms have higher rates of |
| | cyclical mood disorders. Specific agents which may have |
| | therapeutic benefits in patients who exhibit CACNA1C |
| | polymorphisms include hydroxyfasudil, Flunarazine, |
| | Angiotensin receptor blockers |
| | GRIK 4 polymorphisms have been associated with treatment |
| | resistant depression. modulators of the kainate receptor |
| | include Aniracetam and Riluzole. |

Polymorphisms in BDNF may be treated with sigma receptor agonists including DHEA-S, Aniracetam and Tianeptine.

While the methods, kits, assays (and methods for using them) have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A panel assay to determine the presence of SNPs that up-regulate or inhibit CaMKII activity, the panel assay comprising: a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis.

2. The panel assay of claim 1, further comprising an interpretive comment indicating the effect of any identified SNPs on the regulation of CaMKII activity.

3. The panel assay of claim 1, further comprising an interpretive comment suggesting a treatment based on identified SNPs.

4. The panel assay of claim 1, wherein the SNP indicator indicates an SNP that alters the function or expression of the 5HT1a, SERT and BDNF genes in the serotonin metabolism pathway.

5. The panel assay of claim 1, wherein the SNP indicator indicates an SNP that alters the function or expression of the MTHF, TREK, or COMT genes in the dopamine metabolism pathway.

6. The panel assay of claim 1, wherein the SNP indicator indicates an SNP that alters the function or expression of the CACNA1C or GRIK4 genes in the glutamate metabolism pathway.

7. The panel assay of claim 1, wherein the SNP indicator indicates an SNP that alters the function or expression of the TREK, FKBP5 or CACNA1C genes in the hypothalamic pituitary adrenal axis.

8. The panel assay of claim 1, wherein the SNP indicators comprise PCR-based assays.

9. A panel assay to determine the presence of SNPs that up-regulate or inhibit CaMKII activity, the panel assay comprising:
a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and
an interpretive comment indicating the effect of any identified SNPs on the regulation of CaMKII activity.

10. The assay of claim 9, wherein the interpretive comment indicates no effect, up-regulation or down-regulation of CaMKII.

11. The assay of claim 9, further comprising an interpretive comment suggesting a treatment based on identified SNPs.

12. The assay of claim 9, wherein the SNP indicator indicates an SNP that alters the function or expression of the 5HT1a, SERT and BDNF genes in the serotonin metabolism pathway.

13. The assay of claim 9, wherein the SNP indicator indicates an SNP that alters the function or expression of the MTHF, TREK, or COMT genes in the dopamine metabolism pathway.

14. The assay of claim 9, wherein the SNP indicator indicates an SNP that alters the function or expression of the CACNA1C or GRIK4 genes in the glutamate metabolism pathway.

15. The assay of claim 9, wherein the SNP indicator indicates an SNP that alters the function or expression of the TREK, FKBP5 or CACNA1C genes in the hypothalamic pituitary adrenal axis.

16. A panel assay to determine the presence of SNPs that up-regulate or inhibit CaMKII activity, the panel assay comprising:
a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and
an interpretive comment suggesting a treatment based on the identified SNPs.

17. The assay of claim 16, further comprising an interpretive comment indicating the effect of any identified SNPs on the regulation of CaMKII activity.

18. The assay of claim 16, wherein the SNP indicator indicates an SNP that alters the function or expression of the 5HT1a, SERT and BDNF genes in the serotonin metabolism pathway.

19. The assay of claim 16, wherein the SNP indicator indicates an SNP that alters the function or expression of the MTHF, TREK, or COMT genes in the dopamine metabolism pathway.

20. The assay of claim 16, wherein the SNP indicator indicates an SNP that alters the function or expression of the CACNA1C or GRIK4 genes in the glutamate metabolism pathway.

21. The assay of claim 16, wherein the SNP indicator indicates an SNP that alters the function or expression of the TREK, FKBP5 or CACNA1C genes in the hypothalamic pituitary adrenal axis.

22. A kit to determine the presence of SNPs that up-regulate or inhibit CaMKII activity, the kit comprising:
an SNP assay indicating the presence or absence of an SNP that alters the function or expression of a gene in the serotonin metabolism pathway;
an SNP assay indicating the presence or absence of an SNP that alters the function or expression of a gene in the dopamine metabolism pathway;
an SNP assay indicating the presence or absence of an SNP that alters the function or expression of a gene in the glutamate metabolism pathway; and
an SNP assay indicating the presence or absence of an SNP that alters the function or expression of a gene in and the hypothalamic pituitary adrenal axis.

23. The kit of claim 22, wherein the SNP assay indicates an SNP that alters the function or expression of the 5HT1a, SERT and BDNF genes in the serotonin metabolism pathway.

24. The kit of claim 22, wherein the SNP assay indicates an SNP that alters the function or expression of the MTHF, TREK, or COMT genes in the dopamine metabolism pathway.

25. The kit of claim 22, wherein the SNP assay indicates an SNP that alters the function or expression of the CACNA1C or GRIK4 genes in the glutamate metabolism pathway.

26. The kit of claim 22, wherein the SNP assay indicates an SNP that alters the function or expression of the TREK, FKBP5 or CACNA1C genes in the hypothalamic pituitary adrenal axis.

27. The kit of claim 22, further comprising interpretive logic configured to analyze the results of all of the SNP assays and to provide an interpretive comment, wherein the interpretive logic is encoded for processing on a processor.

28. The kit of claim 22, wherein the interpretive comment indicates the effect of any identified SNPs on the regulation of CaMKII activity.

29. The kit of claim 22, wherein the interpretive comment suggests a treatment based on the identified SNPs.

30. The kit of claim 22, wherein the interpretive logic is configured to propose a treatment to inhibit CaMKII when the identified SNPs up-regulate CaMKII, and further wherein the interpretive logic is configured to propose a treatment to preferentially activate CaMKII activity when the identified SNPs down-regulate CaMKII.

31. A method of determining the presence of SNPs that up-regulate or inhibit CaMKII activity in a subject, the method comprising: assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis.

32. The method of claim 31, wherein the sample of the subject's tissue is a blood sample.

33. The method of claim 31, further comprising indicating that CaMKII activity is up-regulated or down-regulated based on the assayed SNPs.

34. The method of claim 31, wherein the step of assaying comprises assaying for an SNP that alters the function or expression of the 5HT1a, SERT and BDNF genes in the serotonin metabolism pathway.

35. The method of claim 31, wherein the step of assaying comprises assaying for an SNP that alters the function or expression of the MTHF, TREK, or COMT genes in the dopamine metabolism pathway.

36. The method of claim 31, wherein the step of assaying comprises assaying for an SNP that alters the function or expression of the CACNA1C or GRIK4 genes in the glutamate metabolism pathway.

37. The method of claim 31, wherein the step of assaying comprises assaying for an SNP that alters the function or expression of the TREK, FKBP5 or CACNA1C genes in the hypothalamic pituitary adrenal axis.

38. A method of determining a treatment for depression in a subject by determining the presence of SNPs that up-regulate or inhibit CaMKII activity in the subject, the method comprising:
assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis;
proposing a treatment for depression based on the presence the SNP detected by assaying the sample.

39. The method of claim 38, wherein the step of assaying comprises assaying for an SNP that alters the function or expression of the 5HT1a, SERT and BDNF genes in the serotonin metabolism pathway.

40. The method of claim 38, wherein the step of assaying comprises assaying for an SNP that alters the function or expression of the MTHF, TREK, or COMT genes in the dopamine metabolism pathway.

41. The method of claim 38, wherein the step of assaying comprises assaying for an SNP that alters the function or expression of the CACNA1C or GRIK4 genes in the glutamate metabolism pathway.

42. The method of claim 38, wherein the step of assaying comprises assaying for an SNP that alters the function or expression of the TREK, FKBP5 or CACNA1C genes in the hypothalamic pituitary adrenal axis.

43. A method of determining a treatment for depression in a subject by determining the presence of SNPs that up-regulate or inhibit CaMKII activity in the subject, the method comprising:
assaying a sample of a subject's tissue for the presence of at least one SNP that alters the function or expression of a gene from each of the serotonin metabolism pathway, the dopamine metabolism pathway, the glutamate metabolism pathway, and the hypothalamic pituitary adrenal axis;
determining the net effect of any SNPs detected by assaying the sample on CaMKII activity; and
proposing a treatment for depression that inhibits CaMKII activity if the net effect is to up-regulate CaMKII activity, or that preferentially activates CaMKII activity if the net effect down-regulates CaMKII activity.

44. The method of claim 43, wherein the step of assaying comprises assaying for an SNP that alters the function or expression of the 5HT1a, SERT and BDNF genes in the serotonin metabolism pathway.

45. The method of claim 43, wherein the step of assaying comprises assaying for an SNP that alters the function or expression of the MTHF, TREK, or COMT genes in the dopamine metabolism pathway.

46. The method of claim 43, wherein the step of assaying comprises assaying for an SNP that alters the function or expression of the CACNA1C or GRIK4 genes in the glutamate metabolism pathway.

47. The method of claim 43, wherein the step of assaying comprises assaying for an SNP that alters the function or expression of the TREK, FKBP5 or CACNA1C genes in the hypothalamic pituitary adrenal axis.
